(19) United States
(12) Patent Application Publication
(10) Pub. No.: US 2006/0069032 A1
(43) Pub. Date: Mar. 30, 2006

(54) Beta-endorphin activity in cosmetics and dermatology

(75) Inventors: Philippe Catroux, Nogent Sur Marne (FR); Maria Dalko, Gif S/Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/281,586

(22) Filed: Nov. 18, 2005

Related U.S. Application Data
(63) Continuation of application No. 10/893,324, filed on Jul. 19, 2004, now abandoned.
(60) Provisional application No. 60/508,285, filed on Oct. 6, 2003.

(30) Foreign Application Priority Data
Jul. 17, 2003 (FR) 0308727

(51) Int. Cl. A61K 38/33

(52) U.S. Cl. 514/15; 514/16; 514/17

(57) ABSTRACT

The subject of the invention is the use of at least one compound chosen from β-endorphin and β-endorphin-mimetic agents on the keratinocytes of the skin, for example in a cosmetic composition to enhance the barrier function of the skin, enhance its resistance to stress and in particular to pollution. It also relates to a method of cosmetic treatment for enhancing the barrier function and/or the moisturization of the skin and/or mucous membranes.

### REFERENCE TO PRIOR APPLICATIONS

This application claims priority to U.S. provisional application 60/508,285 filed Oct. 6, 2003, and to French patent application 0308727 filed Jul. 17, 2003, both incorporated herein by reference.

### FIELD OF THE INVENTION

The invention relates to the use of agents exerting a beta-endorphin-like activity in cosmetics and dermatology. The invention also relates to the uses of the abovementioned agents in a composition or for the preparation of a composition for the skin and/or hair, and to a method for the cosmetic treatment of the skin and/or hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The drawings and description are to be regarded as illustrative in nature, and not as restrictive.

### BACKGROUND OF THE INVENTION

The skin constitutes a barrier against external attacks, in particular chemical, mechanical or infectious attacks, and as such a number of defensive reactions against environmental factors (climate, ultraviolet rays, tobacco and the like) and/or xenobiotic factors, such as for example microorganisms, occur at this level.

The skin consists of two compartments, one which is superficial, the epidermis, and one which is deeper, the dermis, which interact. The natural human epidermis is mainly composed of three types of cells which are the keratinocytes, which form the great majority, the melanocytes and the Langerhans' cells. Each of these cell types contributes, by its specific functions, to the essential role played in the body by the skin, in particular the role of protecting the body from external attacks called “barrier function”.

The epidermis is conventionally divided into a basal layer of keratinocytes which consists of the germinative layer of the epidermis, a so-called prickle cell layer consisting of several layers of polyhedral cells arranged on the germinative layers, one to three so-called granular layers consisting of flattened cells containing distinct cytoplasmic inclusions, the keratohyalin granules and finally the horny layer (or stratum corneum), consisting of a set of layers of keratinocytes at the final stage of their differentiation, called corneocytes.

The dermis provides the epidermis with a solid support. It is also its feeder component. It consists mainly of fibroblasts and an extracellular matrix mainly composed of collagen, elastin and a substance called ground substance. These components are synthesized by the fibroblasts. Leucocytes, mastocytes or tissue macrophages are also found therein. Finally, the dermis is crossed by blood vessels and nerve fibres.

The cohesion between the epidermis and the dermis is provided by the dermoepidermal junction. The equilibrium between the skin barrier and the mucous membranes depends on complex biological mechanisms which involve numerous growth factors, hormones and enzymes. Impairment of the skin barrier can in particular result in a moisturization disorder.

β-Endorphin is a member of the group of “peptides derived from proopiomelanocortin” (POMC) which comprises, inter alia, adrenocorticotropic hormone (ACTH), α-melanocyte-stimulating hormone (α-MSH) and β-lipotropic hormone (β-LPH). β-Endorphin is a peptide of 31 amino acids corresponding to the amino acids sequence 61-91 of β-LPH, which is itself derived from the cleavage of POMC.

POMC and peptides derived from POMC are formed mainly at the central level in the pituitary gland and the hypothalamus. β-Endorphin is secreted more precisely by the melanotropic cells in the anterior lobe of the pituitary gland. These peptides are components of the hypothalamohypophyseal-adrenal axis. After entering into the bloodstream, they are capable of exerting effects on a wide variety of peripheral target tissues. β-Endorphin exerts its biological effects after attachment to opiate-type receptors. There are four types of opiate receptors, called mu, delta, kappa and lambda. β-Endorphin has a high affinity for the mu and delta receptors. The human skin tissue constitutively expresses an active β-endorphin receptor (Bigliardi et al., 1998). β-Endorphin is indeed a specific agonist of the μ-type opiate receptor in the human skin. These receptors are expressed in all the layers of the epidermis with a more pronounced labelling at the level of the basal layers. They have also been identified at the level of the ducts of the sweat glands, of the hair follicles (Weinstein D D et al., J. Invest. Dermatol, 2002, 709-710) and of the melanocytes (Kauser S et al., J. Invest. Dermatol, 2003, 120, 1073-1080).

In addition to their central origin, the peptides derived from POMC are produced by various peripheral tissues (testes, ovaries, liver, kidneys, lungs, gastrointestinal tract, cells of the immune system and the like). At the skin level, it has been shown that some types of cells such as melanocytes, keratinocytes, endothelial cells, Langerhans' cells and dermal fibroblasts are capable of synthesizing ACTH and α-MSH. Stimuli (tumour promoters, interleukin-1, ultraviolet radiation) can in fact increase this secretion under certain experimental conditions. It is described in the literature that various cell types present at the skin level and in particular the human keratinocytes which constitute one of the main components of the epidermis, are capable of synthesizing and secreting beta-endorphin. According to these authors, the β-endorphin produced locally is thought to be capable of exerting local or general biological effects.

Application FR 2 811 228 describes the use of oligosaccharides for stimulating the production of beta-endorphins in the skin; the objective of the authors is to generate a relaxing, soothing, or even an analgesic activity by the release of this peptide in the skin.

The specific role of β-endorphin in the skin and more particularly in the epidermis remains poorly defined. A recent study shows its potential effect in melanogenesis by direct action at the level of the melanocyte. At the level of the epidermal keratinocyte, Nissen J B et al (Exp. Dermatol. 1997, 6, 222-229) indicate that β-endorphin has no effect on epidermal proliferation and differentiation.

### DETAILED DESCRIPTION OF THE PREFERRED EMBODIMENTS

In the present invention, the inventors have demonstrated, completely unexpectedly, that the use of agents exerting a beta-endorphin-like activity promote keratinocyte differentiation and cornification of the keratinocytes, which represents a newly described activity. The latter is favourable for improving the moisturization state of the skin and for combating impairment of the skin barrier which occurs following external stress (effects of atmospheric pollution, ultraviolet radiation and the like) or internal stress termed psychological stress. To demonstrate these effects on the human keratinocyte in culture, the inventors used a model of reconstructed epidermis consisting of keratinocytes in a multilayer undergoing proliferation, differentiation or cornification. It is well known that this in vitro model is suitable for evaluating the effects of an active agent on keratinocyte proliferation and differentiation.

The activity of β-endorphin in this model results in the simultaneous activation of three main factors of terminal epidermal differentiation and of cornification of the keratinocytes. These three factors are calmodulin-like skin protein (CLSP), loricrin and transglutaminase type I (TGK). CLSP is a specific skin calciprotein (Mehul B et al, JID, 2000, 275, 17, 12841-7).

In normal human skin, CLSP is expressed exclusively in the stratum granulosum and in the lower layers of the stratum corneum. It is nowadays recognized as an important factor in keratinocyte differentiation. Loricrin is a basic protein which is a precursor of the horny envelope. It participates in the formation of cornified structures by combining through disulphide bridges and especially through L-glutamine-lysine bonds. It is a major component of the keratohyalin L-granules of the granular layer and it is expressed at a late stage of keratinocyte differentiation. TGK is an enzyme belonging to the transpeptidase family. It is a calcium-dependent enzyme which catalyses the formation of ε(γ-glutamyl)lysine isopeptide bridges between epidermal proteins and therefore promotes the formation of horny envelopes. β-Endorphin therefore exhibits the property of activating 3 main and complementary factors in terminal differentiation of the keratinocyte and its cornification. This specific profile manifests itself here as β-endorphin-like effect.

Accordingly, one subject of the present invention is the use of β-endorphin or of a β-endorphin-like compound, in a method of cosmetic treatment for maintaining and/or strengthening the epidermis, in particular for maintaining the integrity and/or the thickness of various layers of the epidermis, in particular of the stratum corneum, and therefore the barrier function of the epidermis.

Taking into consideration the abovementioned elements, it appears that the best means for reproducing the effects of β-endorphin at the epidermal level is to use active agents designated in the present application “β-endorphin-like”, that is to say inducing in the skin, and more particularly in human keratinocytes, the effects of β-endorphin. It is possible either to use β-endorphin and/or its fragments, or to use active agents mimicking the effects of β-endorphin on the differentiation of the human keratinocyte. β-endorphin, its fragments and/or of an agent capable of mimicking the activity of β-endorphin are referred to collectively herein as “β-endorphin-mimetic agents.” In the invention, one or more such materials may be used together in the same compositon.

The expression active agents (or agents) mimicking the effects of β-endorphin or β-endorphin-mimetic agents for the purposes of the present invention is preferably understood to mean substances capable of inducing the production, by keratinocytes, of at least two of the following three factors: calmodulin-like skin protein (CLSP), loricrin and transglutaminase type I (TGK), preferably of these 3 factors. Advantageously, the active agents mimicking the effects of β-endorphin which are used according to the invention will be determined on a model of keratinocytes in multilayer culture. The β-endorphin-mimetic active agents suitable for carrying out the invention increase the production of CLSP, loricrin and/or TGK by keratinocytes in culture preferably by at least 50% compared with control keratinocytes cultured without the β-endorphin-like product. The β-endorphin fragments useful for carrying out the invention will be preferably β-endorphin-like active agents as defined in the preceding text.

The active fragments of β-endorphin may be for example peptide sequences corresponding to the N- or C-terminal part of beta-endorphin, optionally protected on the acid or amine functional group with an appropriate group. Advantageously, fragments are used which have a length of less than or equal to 10 amino acids and preferably contain at least 2 amino acids, in particular of about 2 to 8 amino acids, for example of 6 to 8 amino acids. The terminal acid functional groups of these fragments may be protected by reactions known to persons skilled in the art, in particular by esterification and/or by formation of an amide group; likewise, the amino-terminal end of the β-endorphin fragment may be protected by acylation, in particular by formation of an acetate group. The fragments obtained by deletion and/or substitution of one or more amino acids of the N- or C-terminal sequences of β-endorphin as defined in the preceding text may also be used in the context of the invention. These fragments may be salified with suitable inorganic or organic acids. There may be mentioned, without limitation, salts such as chloride, sulphate, nitrate, borate, carbonate, gluconate, oxalate, acetate, citrate or oleate.

Examples of such fragments include:

H-Lys-Asn-Ala-His-Lys-Lys-Gly-Gln-OH
Ac-Lys-Asn-Ala-His-Lys-Lys-Gly-Gln-OEt
Ac-Lys-Asn-Ala-His-Lys-Lys-Gly-Gln-NH2
H-Ala-His-Lys-Lys-Gly-Gln-OH
Ac-Ala-His-Lys-Lys -Gly-Gln-QEt
Ac-Ala-His-Lys-Lys-Gly-Gln-OH
H-Lys-Gly-Gln-OH
Ac-Lys-Gly-Gln-OEt
Ac-Lys-Gly-Gln-NH2
H-Tyr-Gly-Gly-Phe-Met-Thr-Ser-Glu-OH
Ac-Tyr-Gly-Gly-Phe-Met-Thr-Ser-Glu-OEt
Ac-Tyr-Gly-Gly-Phe-Met-Thr-Ser-Glu-NH2
H-Tyr-Gly-Gly-Phe-Met-Thr-OH
Ac-Tyr-Gly-Gly-Phe-Met-Thr-OEt
Ac-Tyr-Gly-Gly-Phe-Met-Thr-NH2
H-Tyr-Gly-Gly-Phe-Met-OH
Ac-Tyr-Gly-Gly-Phe-Met-NH2
Ac-Tyr-Gly-Gly-Phe-Met-OEt
H-Tyr-Gly-Gly-OH
Ac-Tyr-Gly-Gly-OEt
Ac-Tyr-Gly-Gly-NH2

Unexpectedly, the inventors have demonstrated that an extract of cocoa bean rich in polyphenols and xanthine bases, in particular in theobromine, has an activity profile which is in every respect comparable to that of β-endorphin.

Accordingly, one of the subjects of the present invention is the use of β-endorphin, of one of its fragments or of an agent capable of mimicking the activity of β-endorphin, on the keratinocytes of the skin, in order to promote moisturization of the skin.

Another aspect of the invention relates to the use of β-endorphin, of one of its fragments or of an agent capable of mimicking the activity of β-endorphin, on the keratinocytes of the skin, in order to improve the barrier function of the skin.

According to yet another aspect, the invention relates to the use of β-endorphin, of one of its fragments and/or of an agent capable of mimicking the activity of β-endorphin, on the keratinocytes of the skin in order to enhance the resistance of the skin to external attacks, in particular to pollution and to polluting agents (such as particles, ozone, nitrogen oxides, sulphur oxides and/or heavy metals such as in particular cobalt, cadmium or mercury) or to free radicals, to sudden variations in atmospheric temperature or relative humidity, to wind, to conditioned air, to mechanical stress, in particular to irritation caused by certain rough fabrics or excessively aggressive hygiene treatments; this use may also be intended to enhance the resistance of the skin to internal stress, in particular to modifications induced by psychological stress or fatigue which have an effect on the surface state of the skin.

The β-endorphin and/or the β-endorphin-like agents according to the invention will thus be particularly useful for stimulating the natural moisturization and brightness of the skin, which appears more radiant and softer, and for enhancing and/or reducing the micro-relief of the skin.

The β-endorphin-like agents which are useful according to the invention can be identified without undue experimentation by one of ordinary skill in view of this disclosure, and may be obtained by chemical synthesis or by extraction from natural sources, in particular from plants, by methods known to persons skilled in the art and in particular with aqueous, alcoholic and/or aqueous-alcoholic solvents. Suitable extracts are in particular extracts containing xanthine bases and polyphenols, in particular flavonoids and anthocyanins.

Advantageously, the β-endorphin-like agent according to the invention is an extract of a plant of the Sterculiaceae family, in particular of Theobroma cacao. There may be mentioned more particularly extracts obtained from shells of cocoa beans, for example by aqueous-glycolic extraction, such as that marketed by the company Solabia under the name Caobromine®. The strength of such extracts are measured as xanthine bases, they contain at least 1%, and in particular at least 1.5% caffeine, and at least 0.01%, in particular at least 0.05% theobromine. They also contain flavonoids and magnesium, the latter element in an amount of about 49-50 ppm.

Such extracts are known as slimming agents.

The subject of the invention is therefore also a method of cosmetic treatment for maintaining or promoting moisturization of the skin and/or the mucous membranes, enhancing the barrier function of the skin and/or the mucous membranes, and promoting resistance of the skin and/or the mucous membranes to external attacks linked to the environment or to modifications caused by intrinsic stress, comprising the application of an effective quantity of β-endorphin, of one of its fragments and/or of an agent capable of mimicking the activity of β-endorphin (collectively “β-endorphin-mimetic agents”) on the keratinocytes of the skin. The agent will be applied to the part of the skin to be treated, in particular to the face, the neck or the hands, daily or several times per day; the application will be repeated every day for a period varying according to the desired effects, generally from 3 to 6 weeks, but may be extended or continued on a continuous basis.

The subject of the invention is also the cosmetic use, in a composition containing a physiologically acceptable medium, of at least one compound chosen from β-endorphin-mimetic agents, as an agent for promoting differentiation and/or cornification of the keratinocytes; in particular it relates to the use, as an agent for maintaining and/or strengthening the epidermis, for maintaining the integrity and/or thickness of the various layers of the epidermis, in particular of the stratum corneum, for maintaining and/or enhancing the barrier function of the skin, and/or for promoting natural moisturization of the skin.

The β-endorphin-mimetic agents may be in particular fragments of β-endorphin, as defined above. β-Endorphin-mimetic agents may be used alone or as mixtures in any proportions.

Another subject of the invention is the use of at least one compound chosen from β-endorphin-mimetic agents on the keratinocytes of the skin for the preparation of a cosmetic and/or dermatological composition intended for combating impairments of the barrier function and/or modifications of the integrity of the stratum corneum.

Preferably, the β-endorphin-mimetic agents are not intended for combating the phenomena of hypersensitization linked to the presence of nickel.

The β-endorphin-mimetic agents according to the invention will be preferably formulated in compositions suitable for topical application to the skin, that is to say in compositions for cosmetic or dermatological use. The quantity will be adjusted by persons skilled in the art according to the active-agent used, but will be generally at least 0.0001% and may for example vary from 0.001% to 10%, in particular from 0.01 to 5% by weight relative to the total weight of the composition. For example in the case of an extract of cocoa bean shell, there will be used from 0.1 to 5% by weight of extract diluted at 3% in the extraction solvent; advantageously, the amounts of this dilution in the composition will be about 0.5 to 2%, more particularly from 1 to 1.5% by weight.

This composition may be more or less fluid and may have the appearance of a white or coloured cream, an ointment, a milk, a lotion, a serum, a paste or a mousse. It may also be provided in solid form, in particular in the form of a stick. It can be used as care product and/or as make-up for the skin.

The composition according to the invention may be provided in any form, for example any of the galenic forms normally used in the cosmetic field, and it may be in particular in the form of an optionally gelled oily solution, an optionally biphasic lotion-type dispersion, an emulsion obtained by dispersing a fatty phase in an aqueous phase (O/W) or conversely (W/O), or a triple emulsion (W/O/W or O/W/O) or a vesicular dispersion of the ionic and/or nonionic type. These compositions are prepared according to the customary methods. It is preferable to use, according to this invention, a composition in the form of an oil-in-water emulsion.

When the composition used according to the invention is an emulsion, the proportion of the fatty phase may range from 5 to 80% by weight, and preferably from 5 to 50% by weight relative to the total weight of the composition. The oils, emulsifiers and coemulsifiers used in the composition in the form of an emulsion are chosen from those conventionally used in the field considered. The emulsifier and coemulsifier are present in the composition in a proportion ranging from 0.3 to 30% by weight, and preferably from 0.5 to 20% by weight relative to the total weight of the composition.

As oils which can be used in the invention, there may be mentioned hydrocarbons of inorganic origin (mineral oil) or of synthetic origin (liquid paraffin, isohexadecane), oils of plant origin (apricot kernel oil, liquid fraction of shea butter, avocado oil, soyabean oil), oils of animal origin (lanolin), synthetic oils (perhydrosqualene, pentaerythrityl tetraoctanoate), silicone oils (cyclopentasiloxane and cyclohexasiloxane) and fluorinated oils (perfluoropolyethers). It is also possible to use, as fats, fatty alcohols (cetyl or stearyl alcohol), fatty acids (stearic acid), waxes (carnauba wax, ozokerite, beeswax).

As emulsifiers and coemulsifiers which can be used in the invention, there may be mentioned for example fatty acid esters of polyethylene glycol such as PEG-100 stearate and PEG-20 stearate and fatty acid esters of glycerol such as glyceryl stearate.

In a known manner, the composition used according to the invention may also contain the usual adjuvants in the cosmetic field, such as hydrophilic or lipophilic gelling agents, hydrophilic or lipophilic active agents, preservatives, antioxidants, solvents, perfumes, fillers, sunscreens, pigments, odour absorbers and colouring matter. The quantities of these various adjuvants are those conventionally used in the field considered, and are for example from 0.01 to 20% of the total weight of the composition. These adjuvants, depending on their nature, may be introduced into the fatty phase, into the aqueous phase or into the lipid vesicles. In any case, these adjuvants, and their proportions, will be preferably chosen so as not to adversely affect the desired properties of the endorphin-mimetic agents on the keratinocytes.

As hydrophilic gelling agents, there may be mentioned in particular carboxyvinyl polymers (carbomer), acrylic copolymers such as acrylate/alkyl acrylate copolymers, polyacrylamides, polysaccharides, natural gums and clays, and, as lipophilic gelling agents, there may be mentioned modified clays such as bentones, metal salts of fatty acids, hydrophobic silica and polyethylenes.

As fillers, there may be mentioned for example polyamide (Nylon) particles in spherical form or in the form of microfibres; microspheres of polymethyl methacrylate; ethylene-acrylate copolymer powders; expanded powders such as hollow microspheres, and in particular the microspheres formed of a terpolymer of vinylidene chloride, acrylonitrile and methacrylate and which are marketed under the name EXPANCEL by the company Kemanord Plast; powders made of natural organic materials such as powders made of starch, in particular maize, wheat or rice starch, which are crosslinked or not, such as the powders made of starch crosslinked with octenyl succinate anhydride; microbeads of silicone resin such as those marketed under the name TOSPEARL by the company Toshiba Silicone; silica; metal oxides such as titanium dioxide or zinc oxide; mica; and mixtures thereof.

The composition used according to the invention may additionally contain other active agents, and in particular at least one compound chosen from: moisturizing agents; depigmenting agents; antiglycation agents; NO-synthase inhibitors; agents stimulating the synthesis of dermal or epidermal macromolecules and/or preventing their degradation; agents stimulating the proliferation of fibroblasts and/or keratinocytes or stimulating the differentiation of keratinocytes; muscle relaxants; tightening agents; antipollution and/or anti-free radical agents; sunscreens; and mixtures thereof.

The expression “moisturizing agent” is understood to mean:
either a compound which acts on the barrier function, so as to maintain the moisturization of the stratum corneum, or an occlusive compound. There may be mentioned ceramides, sphingoid-based compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, pentacyclic triterpenes such as ursolic acid, petroleum jelly and lanolin;
or a compound directly increasing the water content of the stratum corneum, such as trehalose and its derivatives, hyaluronic acid and its derivatives, glycerol, pentanediol, sodium pidolate, serine, xylitol, sodium lactate, polyglycerol acrylate, ectoin and its derivatives, chitosan, oligo- and polysaccharides, cyclic carbonates, N-lauroylpyrrolidonecarboxylic acid and N-α-benzoyl-L-arginine;
or a compound activating the sebaceous glands, such as DHEA, its 7-oxidized and/or 17-alkylated derivatives, sapogenins and vitamin D and its derivatives.

The depigmenting agents which may be incorporated into the composition according to the present invention comprise, for example, the following compounds: kojic acid; ellagic acid; arbutin and its derivatives such as those described in Applications EP-895 779 and EP-524 109; hydroquinone; aminophenol derivatives such as those described in Applications WO 99/10318 and WO 99/32077, and in particular N-cholesteryloxycarbonyl-para-aminophenol and N-ethyloxycarbonyl-para-aminophenol; iminophenol derivatives, in particular those described in Application WO 99/22707; L-2-oxothiazolidine-4-carboxylic acid or procysteine, and its salts and esters; ascorbic acid and its derivatives, in particular ascorbyl glucoside; and extracts of plants, in particular of liquorice, of mulberry and of skull-cap, without this list being limiting.

The expression “antiglycation agent” is understood to mean a compound which prevents and/or reduces glycation of the proteins of the skin, in particular of the proteins of the dermis, such as collagen.

Examples of antiglycation agents are plant extracts of the Ericaceae family, such as an extract of blueberry (Vaccinium angustifolium); ergothioneine and its derivatives; and hydroxystilbenes and their derivatives, such as resveratrol and 3,3′,5,5′,-tetrahydroxystilbene.

Examples of NO-synthase inhibitors suitable for use in the present invention comprise in particular an extract of a plant of the species Vitis vinifera which is marketed in particular by the company Euromed under the name Leucocyanidines de raisins extra, or by the company Indena under the name Leucoselect®, or finally by the company Hansen under the name Extrait de marc de raisin; an extract of a plant of the species Olea europaea which is preferably obtained from olive tree leaves and is marketed in particular by the company VINYALS in the form of a dry extract, or by the company Biologia & Technologia under the trade name Eurol BT; and an extract of a plant of the species Ginkgo biloba which is preferably a dry aqueous extract of this plant sold by the company Beaufour under the trade name Ginkgo biloba extrait standard.

Among the active agents stimulating the macromolecules of the dermis or preventing their degradation, there may be mentioned those which act:
either on the synthesis of collagen, such as extracts of Centella asiatica; asiaticosides and derivatives; ascorbic acid or vitamin C and its derivatives; synthetic peptides such as iamin, biopeptide CL or palmitoyloligopeptide marketed by the company SEDERMA; peptides extracted from plants, such as the soyabean hydrolysate marketed by the company COLETICA under the trade name Phytokine®; and plant hormones such as auxins and lignans;
or on the synthesis of elastin, such as the extract of Saccharomyces cerevisiae marketed by the company LSN under the trade name Cytovitin®; and the extract of the alga Macrocystis pyrifera marketed by the company SECMA under the trade name Kelpadelie
or on the synthesis of glycosaminoglycans, such as the product of fermentation of milk by Lactobacillus vulgaris, marketed by the company BROOKS under the trade name Biomin yogourth®; the extract of the brown alga Padina pavonica marketed by the company ALBAN MÜLLER under the trade name HSP3®; and the extract of Saccharomyces cerevisiae available in particular from the company SILAB under the trade name Firmalift® or from the company LSN under the trade name Cytovitin®;
or on the synthesis of fibronectin, such as the extract of the zooplankton Salina marketed by the company SEPORGA under the trade name GP4G®; the yeast extract available in particular from the company ALBAN MULLER under the trade name Drieline®; and the palmitoyl pentapeptide marketed by the company SEDERMA under the trade name Matrixil®;
or on the inhibition of metalloproteinases (MMP) such as more particularly MMP 1, 2, 3, 9. There may be mentioned: retinoids and derivatives, oligopeptides and lipopeptides, lipoamino acids, the malt extract marketed by the company COLETICA under the trade name Collalift®; extracts of blueberry or of rosemary; lycopene; isoflavones, their derivatives or plant extracts containing them, in particular extracts of soyabean (marketed for example by the company ICHIMARU PHARCOS under the trade name Flavosterone SB®), of red clover, of flax, of kakkon or of sage;
or on the inhibition of serine proteases such as leukocyte elastase or cathepsin G. There may be mentioned: the peptide extract of seeds of a legume (Pisum sativum) marketed by the company LSN under the trade name Parelastyl®; heparinoids; and pseudodipeptides such as {2-[acetyl(3-trifluoromethyl-phenyl)amino]-3-methylbutyrylamino}acetic acid.

Among the active agents stimulating epidermal macromolecules such as fillagrin and keratins, there may be mentioned in particular the lupin extract marketed by the company SILAB under the trade name Structurine®; the beech Fagus sylvatica bud extract marketed by the company GATTEFOSSE under the trade name Gatuline®; and the zooplankton Salina extract marketed by the company SEPORGA under the trade name GP4G®.

Agents stimulating the proliferation of fibroblasts which can be used in the composition according to the invention may be chosen for example from plant proteins or polypeptides, extracted in particular from soyabean (for example a soyabean extract marketed by the company LSN under the name Eleseryl SH-VEG 8® or marketed by the company SILAB under the trade name Raffermine®; and plant hormones such as giberrellins and cytokinins.

The agents stimulating proliferation of the keratinocytes, which can be used in the composition according to the invention, comprise in particular retinoids such as retinol and its esters, of which retinyl palmitate; phloroglucinol; nut cake extracts marketed by the company GATTEFOSSE; and Solanum tuberosum extracts marketed by the company SEDERMA.

The agents stimulating differentiation of the keratinocytes comprise for example minerals such as calcium; the lupin extract marketed by the company SILAB under the trade name Photopreventine®; sodium beta-sitosteryl sulphate marketed by the company SEPORGA under the trade name Phytocohesine®; and the maize extract marketed by the company SOLABIA under the trade name Phytovityl®; and lignans such as secoisolariciresinol.

The composition according to the invention may comprise skin-relaxing agents, among which there may be mentioned in particular alverine and its salts, in particular alverine citrate, sapogenins such as diosgenin and natural extracts containing them (such as Wild Yam extracts), certain carbonylated secondary and tertiary amines, organic or inorganic metal salts, in particular manganese gluconate, adenosine, and the hexapeptide argireline R marketed by the company LIPOTEC. Certain perfuming compositions with a skin-relaxing effect may also be mentioned.

The expression “tightening agent” is understood to mean a compound capable of exerting tensile stress on the skin, which has the effect of temporarily attenuating the irregularities of the skin surface, such as wrinkles and fine lines.

The expression “antipollution agent” is understood to mean any compound capable of trapping ozone, mono- or polycyclic aromatic compounds such as benzopyrene and/or heavy metals such as cobalt, mercury, cadmium and/or nickel. The expression “anti-free radical agent” is understood to mean any compound capable of trapping free radicals.

As ozone-trapping agents which can be used in the composition according to the invention, there may be mentioned in particular vitamin C and its derivatives including ascorbyl glucoside; phenols and polyphenols, in particular tannins, ellagic acid and tannic acid; epigallocatechin and natural extracts containing it; olive tree leaf extracts; tea, in particular green tea, extracts; anthocyanins; rosemary extracts; phenol acids, in particular chlorogenic acid; stilbenes, in particular resveratrol; derivatives of sulphur amino acids, in particular S-carboxymethyl-cysteine; ergothioneine; N-acetylcysteine; chelating agents such as N,N′-bis(3,4,5-trimethoxybenzyl)-ethylenediamine or one of its salts, metal complexes or esters; carotenoids such as crocetin; and various raw materials such as the mixture of arginine, histidine ribonucleate, mannitol, adenosine triphosphate, pyridoxine, phenylalanine, tyrosine and hydrolysed RNA marketed by Laboratoires Sérobiologiques under the trade name CPP LS 2633-12F®, the water-soluble fraction of maize marketed by the company SOLABIA under the trade name Phytovityl®, the mixture of fumitory extract and lemon extract marketed under the name Unicotrozon C-49® by the company Induchem, and the mixture of extracts of ginseng, apple, peach, wheat and barley sold by the company PROVITAL under the trade name Pronalen Bioprotect®.

As agents for trapping mono- or polycyclic aromatic compounds which may be used in the composition according to the invention, there may be mentioned in particular tannins such as ellagic acid; indole derivatives, in particular 3-indolecarbinol; tea, in particular green tea, extracts, water hyacinth or Eichhornia crassipes extracts; and the water-soluble fraction of maize marketed by the company SOLABIA under the trade name Phytovityl®.

Finally, as heavy metal trapping agents which can be used in the composition according to the invention, there may be mentioned in particular chelating agents such as EDTA, the pentasodium salt of ethylenediaminetetramethylenephosphonic acid, and N,N′-bis(3,4,5-trimethoxybenzyl)ethylenediamine or one of its salts, metal complexes or esters; phytic acid; chitosan derivatives; tea, in particular green tea, extracts; tannins such as ellagic acid; sulphur amino acids such as cysteine; water hyacinth (Eichhornia crassipes) extracts; and the water-soluble fraction of maize marketed by the company SOLABIA under the trade name Phytovityl®.

The anti-free radical agents which can be used in the composition according to the invention additionally comprise certain antipollution agents mentioned above, vitamin E and its derivatives such as tocopheryl acetate; bioflavonoids; coenzyme Q10 or ubiquinone; certain enzymes such as catalase, superoxide dismutase, lactoperoxidase, glutathione peroxidase and quinone reductases; glutathione; benzylidenecamphor; benzylcyclanones; substituted naphthalenones; pidolates; phytantriol; gamma-oryzanol; lignans; and melatonin.

The composition according to the invention may also contain UVA and/or UVB screening agents or photoprotective agents, in the form of organic and/or inorganic compounds, which are water-soluble or fat-soluble or which are insoluble in commonly used cosmetic solvents.

The organic photoprotective agents are chosen in particular from anthranilates; cinnamic derivatives; dibenzoylmethane derivatives; salicylic derivatives, camphor derivatives; triazine derivatives such as those described in U.S. Pat. No. 4,367,390, EP 863145, EP 517104, EP 570838, EP 796851, EP 775698, EP 878469, EP 933376, EP 507691, EP 507692, EP 790243 and EP 944624; benzophenone derivatives; β,β-diphenyl acrylate derivatives; benzotriazole derivatives; benzalmalonate derivatives; benzimidazole derivatives; imidazolines; bis-benzoazolyl derivatives as described in Patents EP 669323 and U.S. Pat. No. 2,463,264; p-aminobenzoic acid (PABA) derivatives; methylenebis(hydroxyphenyl-benzotriazole) derivatives as described in Applications U.S. Pat. No. 5,237,071, U.S. Pat. No. 5,166,355, GB 2303549, DE 19726184 and EP 893119; screening polymers and screening silicones such as those described in particular in Application WO 93/04665; dimers derived from α-alkylstyrene such as those described in Patent Application DE 19855649; 4,4-diarylbutadienes such as those described in Applications EP 0967200, DE 19746654, DE 19755649, EP-A-1008586, EP 1133980 and EP 133981 and mixtures thereof.

As examples of photoprotective agents which are active in UV-A and/or UV-B, there may be mentioned those designated below under their INCI name:
para-aminobenzoic acid derivatives, including the following: PABA, Ethyl PABA, Ethyl Dihydroxypropyl PABA, Ethylhexyl Dimethyl PABA sold in particular under the name “ESCALOL 507” by ISP, Glyceryl PABA, PEG-25 PABA sold under the name “UVINUL P25” by BASF,
salicylic derivatives, including the following: Homosalate sold in particular under the name “NEO HELIOPAN OS” by Haarmann and REIMER, Dipropyleneglycol Salicylate sold in particular under the name “DIPSAL” by SCHER, TEA Salicylate, sold in particular under the name “NEO HELIOPAN TS” by Haarmann and REIMER,
dibenzoylmethane derivatives, including the following: Butyl Methoxydibenzoylmethane sold in particular under the trade name “PARSOL 1789” by HOFFMANN LA ROCHE, Isopropyl Dibenzoylmethane,
cinnamic derivatives, including the following: Ethylhexyl Methoxycinnamate sold in particular under the trade name “PARSOL MCX” by HOFFMANN LA ROCHE, Isopropyl Methoxy cinnamate, Isoamyl Methoxy cinnamate sold in particular under the trade name “NEO HELIOPAN E 1000” by HAARMANN and REIMER, Cinoxate, DEA Methoxycinnamate, Diisopropyl Methylcinnamate, Glyceryl Ethylhexanoate Dimethoxycinnamate,
β,β′-diphenyl acrylate derivatives, including the following: Octocrylene sold in particular under the trade name “UVINUL N539” by BASF, Etocrylene, sold in particular under the trade name “UVINUL N35” by BASF,
benzophenone derivatives, including the following: Benzophenone-1 sold under the trade name “UVINUL 400” by BASF, Benzophenone-2 sold under the trade name “UVINUL D50” by BASF, Benzophenone-3 or Oxybenzone, sold under the trade name “UVINUL M40” by BASF, Benzophenone-4 sold under the trade name “UVINUL MS40” by BASF, Benzophenone-5, Benzophenone-6 sold under the trade name “Helisorb 11” by Norquay, Benzophenone-8 sold under the trade name “Spectra-Sorb UV-24” by American Cyanamid, Benzophenone-9 sold under the trade name “UVINUL DS-49” by BASF, Benzophenone-12, and n-hexyl 2-(4-diethylamino-2-hydroxybenzoyl)benzoate,
benzylidenecamphor derivatives, including the following: 3-Benzylidene camphor, 4-Methylbenzylidene camphor sold under the name “EUSOLEX 6300” by MERCK, Benzylidene Camphor Sulphonic Acid, Camphor Benzalkonium Methosulphate, Terephthalylidene Dicamphor Sulphonic Acid, Polyacrylamidomethyl Benzylidene Camphor,
benzimidazole derivatives, including the following: Phenylbenzimidazole Sulphonic Acid sold in particular under the trade name “EUSOLEX 232” by MERCK, Disodium Phenyl Dibenzimidazole Tetra-sulphonate sold under the trade name “NEO HELIOPAN AP” by Haarmann and REIMER,
triazine derivatives, including the following: Anisotriazine sold under the trade name “TINOSORB S” by CIBA SPECIALTY CHEMICALS, Ethylhexyl triazone sold in particular under the trade name “UVINUL T150” by BASF, Diethylhexyl Butamido Triazone sold under the trade name “UVASORB HEB” by SIGMA 3V, and 2,4,6-Tris(diisobutyl 4′-aminobenzalmalonate)-s-triazine,
benzotriazole derivatives, including the following: Drometrizole Trisiloxane sold under the name “Silatrizole” by RHODIA CHIMIE, Methylene bis-Benzotriazolyl Tetramethylbutylphenol, sold in solid form under the trade name “MIXXIM BB/100” by FAIRMOUNT CHEMICAL or in micronized form in aqueous dispersion under the trade name “TINOSORB M” by CIBA SPECIALTY CHEMICALS,
anthranilic derivatives, including Menthyl anthranilate sold under the trade name “NEO HELIOPAN MA” by Haarmann and REIMER,
imidazoline derivatives, including Ethylhexyl Dimethoxybenzylidene Dioxoimidazoline Propionate,
benzalmalonate derivatives, including polyorganosiloxane with benzalmalonate functional groups sold under the trade name “PARSOL SLX” by HOFFMANN LA ROCHE,
4,4-diarylbutadiene derivatives, including 1,1-dicarboxy(2,2′-dimethylpropyl)-4,4-diphenyl-butadiene,
and mixtures thereof.

The organic photoprotective agents most particularly preferred are chosen from the following compounds: Ethylhexyl Salicylate, Ethylhexyl Methoxycinnamate, Octocrylene, Phenylbenzimidazole Sulphonic Acid, Benzophenone-3, Benzophenone-4, Benzophenone-5,4-Methylbenzylidene camphor, Terephthalylidene Dicamphor Sulphonic Acid, Disodium Phenyl Dibenzimidazole Tetra-sulphonate, 2,4,6-tris(diisobutyl 4′-aminobenzalmalonate)-s-triazine, Anisotriazine, Ethylhexyl triazone, Diethylhexyl Butamido Triazone, Methylene bis-Benzotriazolyl-Tetramethylbutylphenol, Drometrizole Trisiloxane, 1,1-dicarboxy(2,2′-dimethylpropyl)-4,4-diphenylbutadiene and mixtures thereof.

The inorganic photoprotective agents are chosen from pigments or alternatively nanopigments (mean primary particle size: generally between 5 nm and 100 nm, preferably between 10 nm and 50 nm) of metal oxides, coated or otherwise, such as for example nanopigments of titanium oxide (amorphous or crystallized in rutile and/or anatase from), of iron oxide, of zinc oxide, of zirconium oxide or of cerium oxide which are all UV photoprotective agents well known per se. Conventional coating agents are moreover alumina and/or aluminium stearate. Such coated or uncoated nanopigments of metal oxides are described in particular in Patent Applications EP518772 and EP518773.

The photoprotective agents are generally present in the compositions according to the invention in proportions ranging from 0.1 to 20% by weight relative to the total weight of the composition, and preferably ranging from 0.2 to 15% by weight relative to the total weight of the composition.

The examples which follow are intended to illustrate the invention.

### EXAMPLE 1

### Demonstration of the Activity on the Keratinocytes

Experimental Studies

The study was carried out on a model of reconstructed epidermis SkinEthic. Upon receiving, the epidermes were precultured for 24 hours in SkinEthic medium. The culture medium was then replaced with medium containing the test product (two epidermes per concentration tested), and then the epidermes were cultured for 24 hours at 37° C. and 5% CO₂. A batch of 2 epidermes was not treated. The culture supernatants were then removed and the epidermes were rinsed with a PBS solution, cut and deposited in sterile RNA-free tubes in the presence of Tri-Reagent (Sigma T9424) and then immediately frozen at −80° C. The markers whose expression was sought are presented in the table below.

The pairs of primers which were used in this study allow the amplification of the following specific fragments:

| Markers selected | Abbreviation | Genbank access |
|---|---|---|
| Transglutaminase 1 | TGK | X57974 |
| Loricrin | LORI | M61120 |
| Calmodulin-like skin protein | CLSP | AF172852 |

The reverse transcription was carried out in the following manner:
extraction of the total RNA of each epidermis with the aid of Tri-Reagent according to the protocol recommended by the supplier, and then a new extraction with chloroform and precipitation with isopropanol.
removal of the traces of potentially contaminating DNA by treating with the DNA-free system (Ambion)
carrying out of the reaction of reverse transcription of the mRNA in the presence of the oligo(dT) primer and of the enzyme Superscript II (Gibco)
quantification, by fluorescence, of the synthesized cDNA and adjustment of the concentrations of 50 ng/ml.

The PCR reactions (polymerase chain reactions) were carried out by quantitative PCR with the “Light cycler” system (Roche Molecular Systems Inc.) according to the procedures recommended by the supplier.

The PCR conditions were the following: activation of 10 min at 95° C., PCR reactions (10 sec at 95° C., 5 sec at 64° C. and 35 sec at 72° C.) 40 cycles, fusion of 5 sec at 95° C. and then 5 sec at 60° C.

The incorporation of fluorescence into the amplified DNA was measured continuously during the PCR cycles. This system makes it possible to obtain fluorescence measurement curves as a function of the PCR cycles and to thereby evaluate a relative expression value for each marker. The RE (relative expression) value is expressed in arbitrary units according to the following formula: (1/2^{number of cycles})×10⁶.

Results

The results were expressed relative to the quantity of actin messenger (reference gene) and are represented, per marker, in the following tables.

Transglutaminase I (TGK) Marker

| | | RE*actin | RE*TGK | TGK/ | |
|---|---|---|---|---|---|
| Treatment | Concentration | (UA) | (UA) | actin | % control |
| Control | — | 1.11 | 0.094 | 0.085 | 100 |
| Beta- | 0.01 μM | 1.11 | 0.165 | 0.149 | 176 |
| endorphin | | | | | |
| Cocoa bean | 0.1% | 1.10 | 0.22 | 0.201 | 238 |
| extract | | | | | |

Caobromine, at the highest concentration, causes a marked increase in the expression of the TGK messenger. β-endorphin, at physiological concentration, induces an effect of the same type, although slightly less pronounced.

Marker Camodulin-Like Skin Protein (CLSP)

| | | RE*actin | RE*CLSP | CLSP/ | % |
|---|---|---|---|---|---|
| Treatment | Concentration | (UA) | (UA) | actin | control |
| Control | — | 1.04 | 0.0574 | 0.0552 | 100 |
| Beta- | 0.01 μM | 1.07 | 0.1032 | 0.0961 | 174 |
| endorphin | | | | | |
| Control | — | 0.94 | 0.06 | 0.061 | 100 |
| Cocoa bean | 0.1% | 0.94 | 0.17 | 0.178 | 291 |
| extract | | | | | |

The two products, regardless of the concentration used, induce an increase in the expression of the CLSP messenger.

Loricrin Marker

| | | RE*actin | RE*LORI | LORI/ | % |
|---|---|---|---|---|---|
| Treatment | Concentration | (UA) | (UA) | actin | control |
| Control | — | 1.09 | 6.19 | 5.67 | 100 |
| Beta- | 0.01 μM | 1.11 | 11.24 | 10.2 | 179 |
| endorphin | | | | | |
| Cocoa bean | 0.1% | 1.06 | 16.13 | 15.19 | 268 |
| extract | | | | | |

β-Endorphin at physiological concentration and caobromine at the two concentrations tested stimulate the expression of this marker.

### EXAMPLE 2

### Preparation of an O/W Emulsion O/W Emulsion

Phase A (oily phase):

| | |
|---|---|
| Mixture of arachidyl polyglucoside and | 1.5% |
| arachidyl and behenyl alcohols (15/85) | |
| (Montanov 202 from the company SEPPIC) | |
| Shea butter | 1% |
| Mixture of glyceryl monodistearate and | 0.5% |
| potassium stearate (Tegin from the | |
| company Goldschmidt) | |
| Caprylic/capric triglycerides | 4% |
| Cyclopentadimethylsiloxane | 6% |

Phase B (aqueous phase)

| | |
|---|---|
| Glycerine | 5% |
| Acrylamide copolymer (Hostacerin AMPS ® s | 1% |
| from Clariant) | |
| Preservative | qs |
| Water | qs 100% |

Phase C

| | |
|---|---|
| Extract of shell of cocoa beans | 1% |
| (Theobroma cacao) in Stab. Butylene | |
| Glycol/Water (that is 3.3% of active | |
| material) | |

Phase D

| | |
|---|---|
| Perfume | qs |

Procedure:

The phases are prepared, phases A and B with the use of heat (about 80° C.) and phase A is incorporated into phase B, with stirring. After cooling to 40° C., phase C and phase D are added, with low shearing.

### EXAMPLE 3

### Composition for Dry Skins

The composition is prepared according to the following formula:

| | |
|---|---|
| UV-screening agent | 0.1000% |
| pigments and pearlescent agents | 0.2000% |
| ethyl alcohol | 3.5000% |
| sequesterant | 0.1000% |
| tocopheryl acetate | 0.1000% |
| oxyethylenated methylglucose sesquistearate | 2.5000% |
| Vegetable oils | 2.5000% |
| sodium hylauronate | 0.0200% |
| glycerine | 7.0000% |
| preservatives | 0.8000% |
| 4-methylesculetol sodium monoethanoate | 0.1000% |
| Perfume | 0.1500% |
| cyclohexadimethylsiloxane | 6.0000% |
| triglycerides of caprylic-capric acids | 6.0000% |
| Fillers | 2.0000% |
| thickeners and gelling agents | 7.0000% |
| block copolymers [polydimethylsioxane] | 0.5000% |
| [ethyl] [polydimethylsiloxane] | |
| extract of shell of cocoa beans (Theobroma | 1.0000% |
| cacao) in Water/Butylene Glycol | |
| Water | qs 100% |

### EXAMPLE 4

### Effect on Moisturization

The composition of Example 3 is evaluated according to the following protocol:
Number of subjects: 25 subjects
Method of evaluation: corneometer
Measurement Time:
   T0, before the first application of the product to the forearm
   T4 weeks, after the last application of the product to the forearm dating from the previous day

Results:

| | Control | Treated |
|---|---|---|
| T0 | 29.4 +/− 6.0 | 29.4 +/− 6.2 |
| T4 w | 29.2 +/− 5.9 | 40.8 +/− 8.7 |

Gain in Moisturization:
T4w: +39.5% p<0.0001 (S)

### CONCLUSION

Under the study conditions, and compared with a control zone, the treatment according to the invention has a significant moisturizing effect after 4 weeks of twice daily use, the gain in moisturization is about +39.5%.

The above written description of the invention provides a manner and process of making and using it such that any person skilled in this art is enabled to make and use the same, this enablement being provided in particular for the subject matter of the appended claims and including the use of at least one β-endorphin-mimetic agent on the keratinocytes of the skin, in a cosmetic composition for enhancing the barrier function of the skin, and/or enhancing moisturization of the skin, and/or strengthening the resistance of the skin to internal and/or external stress, and/or strengthening the resistance of the skin to pollution, and/or to enhance the brightness of the skin.

As used herein, the phrase “a composition comprising an effective amount of at least one β-endorphin-mimetic agent” includes compositions with multiple β-endorphin-mimetic agents, none of which, by themselves, is present in an amount sufficient to be effective on its own but together provide the desired benefit.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for enhancing the barrier function of the skin, and/or enhancing moisturization of the skin, and/or strengthening the resistance of the skin to internal and/or external stress, and/or strengthening the resistance of the skin to pollution, and/or to enhance the brightness of the skin,
comprising applying to skin in need thereof a composition comprising an effective amount of at least one β-endorphin-mimetic agent to enhance the barrier function of the skin, and/or enhance moisturization of the skin, and/or strengthen the resistance of the skin to internal and/or external stress, and/or strengthen the resistance of the skin to pollution, and/or to enhance the brightness of the skin.

2. The method according to claim 1, wherein said method is a method to enhance moisturization of the skin, and wherein said composition comprises an effective amount of at least one β-endorphin-mimetic agent to enhance moisturization.

3. The method according to claim 1, wherein said method is a method to strengthen the resistance of the skin to internal and/or external stress, and wherein said composition comprises an effective amount of at least one β-endorphin-mimetic agent to strengthen the resistance of the skin to internal and/or external stress.

4. The method according to claim 1, wherein said method is a method to strengthen the resistance of the skin to pollution, and wherein said composition comprises an effective amount of at least one β-endorphin-mimetic agent to strengthen the resistance of the skin to pollution.

5. The method according to claim 1, wherein said method is a method to enhance the brightness of the skin, and wherein said composition comprises an effective amount of at least one β-endorphin-mimetic agent to enhance the brightness of the skin.

6. The method according to claim 1, wherein said method is a method to enhance the barrier function of the skin, and wherein said composition comprises an effective amount of at least one β-endorphin-mimetic agent to enhance the barrier function of the skin.

7. The method according to claim 1, wherein the at least one β-endorphin-mimetic agent activates the production by the keratinocytes of at least two factors selected from the group consisting of calmodulin-like skin protein (CLSP), loricrin and transglutaminase type I (TGK).

8. The method according to claim 1, wherein the composition comprises at least one compound selected from the group consisting of:
H-Lys-Asn-Ala-His-Lys-Lys-Gly-Gln-OH
Ac-Lys-Asn-Ala-His-Lys-Lys-Gly-Gln-OEt
Ac-Lys-Asn-Ala-His-Lys-Lys-Gly-Gln-NH2
H-Ala-His-Lys-Lys-Gly-Gln-OH
Ac-Ala-His-Lys-Lys-Gly-Gln-OEt
Ac-Ala-His-Lys-Lys-Gly-Gln-OH
H-Lys-Gly-Gln-OH
Ac-Lys-Gly-Gln-OEt
Ac-Lys-Gly-Gln-NH2
H-Tyr-Gly-Gly- Phe-Met-Thr-Ser-Glu-OH
Ac-Tyr-Gly-Gly-Phe-Met-Thr-Ser-Glu-OEt
Ac-Tyr-Gly-Gly-Phe-Met-Thr-Ser-Glu-NH2
H-Tyr-Gly-Gly-Phe-Met-Thr-OH
Ac-Tyr-Gly-Gly-Phe-Met-Thr-OEt
Ac-Tyr-Gly-Gly-Phe-Met-Thr-NH2
H-Tyr-Gly-Gly-Phe-Met-OH
Ac-Tyr-Gly-Gly-Phe-Met-NH2
Ac-Tyr-Gly-Gly-Phe-Met-OEt
H-Tyr-Gly-Gly-OH
Ac-Tyr-Gly-Gly-OEt
and
Ac-Tyr-Gly-Gly-NH2.

9. The method according to claim 1, wherein the composition comprises at least one aqueous, alcoholic and/or aqueous-alcoholic extract of part of the plant Theobroma cacao.

10. The method according to claim 1, wherein the composition comprises an aqueous-alcoholic extract of cocoa bean shell.

11. The method according to claim 1, wherein said composition further comprises at least one compound selected from the group consisting of moisturizing agents; depigmenting agents; antiglycation agents; NO-synthase inhibitors; agents stimulating the synthesis of dermal or epidermal macromolecules and/or preventing their degradation; agents stimulating the proliferation of fibroblasts and/or keratinocytes or stimulating the differentiation of keratinocytes; muscle relaxants; tightening agents; antipollution and/or anti-free radical agents; sunscreens, and mixtures thereof.

12. The method of claim 1, wherein said composition comprises β-endorphin-mimetic agent β-endorphin.

13. The method of claim 2, wherein said composition comprises β-endorphin-mimetic agent β-endorphin.

14. The method of claim 3, wherein said composition comprises β-endorphin-mimetic agent β-endorphin.

15. The method of claim 4, wherein said composition comprises β-endorphin-mimetic agent β-endorphin.

16. The method of claim 5, wherein said composition comprises β-endorphin-mimetic agent β-endorphin.

17. The method of claim 6, wherein said composition comprises β-endorphin-mimetic agent β-endorphin.

18. The method of claim 1, wherein said composition comprises at least one compound selected from the group consisting of β-endorphin fragments and plant extracts.

19. The method of claim 1, wherein said composition comprises an extract of a plant of the Sterculiaceae family


SEQUENCE LISTING (15 sequences)

SEQ ID NO: 1 - protein, 8 aa, Artificial Sequence
  Synthetic Peptide
KNAHKKGQ

SEQ ID NO: 2 - protein, 8 aa, Artificial Sequence
  Synthetic Peptide
KNAHKKGQ

SEQ ID NO: 3 - protein, 8 aa, Artificial Sequence
  Synthetic Peptide
KNAHKKGQ

SEQ ID NO: 4 - protein, 6 aa, Artificial Sequence
  Synthetic Peptide
AHKKGQ

SEQ ID NO: 5 - protein, 6 aa, Artificial Sequence
  Synthetic Peptide
AHKKGQ

SEQ ID NO: 6 - protein, 6 aa, Artificial Sequence
  Synthetic Peptide
AHKKGQ

SEQ ID NO: 7 - protein, 8 aa, Artificial Sequence
  Synthetic Peptide
YGGFMTSE

SEQ ID NO: 8 - protein, 8 aa, Artificial Sequence
  Synthetic Peptide
YGGFMTSE

SEQ ID NO: 9 - protein, 8 aa, Artificial Sequence
  Synthetic Peptide
YGGFMTSE

SEQ ID NO: 10 - protein, 6 aa, Artificial Sequence
  Synthetic Peptide
YGGFMT

SEQ ID NO: 11 - protein, 6 aa, Artificial Sequence
  Synthetic Peptide
YGGFMT

SEQ ID NO: 12 - protein, 6 aa, Artificial Sequence
  Synthetic Peptide
YGGFMT

SEQ ID NO: 13 - protein, 5 aa, Artificial Sequence
  Synthetic Peptide
YGGFM

SEQ ID NO: 14 - protein, 5 aa, Artificial Sequence
  Synthetic Peptide
YGGFM

SEQ ID NO: 15 - protein, 5 aa, Artificial Sequence
  Synthetic Peptide
YGGFM